(19) United States
(12) Patent Application Publication
(10) Pub. No.: US 2025/0186144 A1
(43) Pub. Date: Jun. 12, 2025

(54) DEVICE AND METHOD FOR TRACKING MOVEMENT OF ROBOT IN ROBOT-ASSISTED SURGERY

(71) Applicant: ORTHOSOFT ULC, Montreal (CA)

(72) Inventor: Daniel SALVIANO, Montreal (CA)

(21) Appl. No.: 18/972,190

(22) Filed: Dec. 6, 2024

Related U.S. Application Data
(60) Provisional application No. 63/608,329, filed on Dec. 11, 2023.

(51) Int. Cl. A61B 34/30; A61B 90/00; A61B 90/30

(52) U.S. Cl. CPC A61B 34/30; A61B 90/30; A61B 90/36; A61B 2034/304; A61B 2034/305; A61B 2560/0223

(57) ABSTRACT

A surgical robot for computer-assisted surgery may include a floor-mounted base. A robotic arm is supported by the floor-mounted base. A calibration device is on the floor-mounted base. The calibration device may include a light source, an image capture component, lenses for directing light from the light source onto the floor and for directing light reflected and/or backscattered from the floor onto the image capture component. A calibration module is coupled to the image capture component, the calibration module for quantifying a movement of the floor-mounted base relative to the floor using data from the image capture component associated with the light reflected and/or backscattered from the floor.

### CROSS-REFERENCE TO RELATED APPLICATION

The present application claims the priority of U.S. Patent Application No. 63/608,329, filed on Dec. 11, 2023, the content of which is incorporated herein by reference.

### TECHNICAL FIELD

The present application relates to navigation in robotic-assisted surgery, such as orthopedic surgery, and in computer-assisted surgery using optical navigation.

### BACKGROUND

Navigation technologies are commonly used in computer-assisted surgery. Navigation technologies may involve the use of cameras or like imaging devices that may track objects such as patient tissues and tools during a surgical or dental procedure. The objects may consequently be tracked in a global referential system for the relative positions and orientations of the objects to be calculable in real time. This allows for example an operator to have guidance on a resection of a bone.

Such procedures commonly imply high levels of accuracy and precision, whereby various calibration steps may be required before and during the procedures. The camera or like imaging device and the robot are at the core of the global referential system, due to their fixed position. However, it may occur that medical or dental personnel come into contact with the robot and/or camera or that other events move the robot and/or camera, and this may have the effect of de-calibrating the robot and/or camera relative to the global referential system. The procedure may consequently be halted for the various objects to be recalibrated. This may for example lengthen the duration of surgery.

### SUMMARY

In accordance with a first aspect of the present disclosure, there is provided a surgical robot for computer-assisted surgery, comprising: a floor-mounted base; a robotic arm supported by the floor-mounted base; and a calibration device on the floor-mounted base or robotic arm, the calibration device including a light source configured for projecting light on a floor, an image capture component configured for capturing light reflected and/or backscattered from the floor, and a calibration module having a processing unit and a non-transitory computer-readable memory communicatively coupled to the processing unit and comprising computer-readable program instructions executable by the processing unit for quantifying a movement of the floor-mounted base relative to the floor using data from the image capture component associated with the light reflected and/or backscattered from the floor.

Further in accordance with the first aspect, for example, the calibration module is for quantifying a movement of the floor-mounted base in translation relative to a plane of the floor, and in rotation relative to a yaw axis of the base.

Still further in accordance with the first aspect, for example, the calibration device further includes an inertial sensor, the calibration module coupled to inertial sensor for quantifying a variation of orientation of the floor-mounted base relative to the floor using data from the inertial sensor, the variation of orientation being relative to a roll axis and/or to a pitch axis of the base.

Still further in accordance with the first aspect, for example, at least one lens may be provided for directing light from the light source onto the floor and for directing light reflected and/or backscattered from the floor onto the image capture component.

Still further in accordance with the first aspect, for example, one of the at least one lens is configured to be at most 6 inches from the floor.

Still further in accordance with the first aspect, for example, the calibration device is mounted to the floor-mounted base.

Still further in accordance with the first aspect, for example, the light source and/or the image capture component is at most 6 inches from the floor.

Still further in accordance with the first aspect, for example, the light source is a LED light source.

Still further in accordance with the first aspect, for example, the image capture component is an image pixel array.

Still further in accordance with the first aspect, for example, the floor-mounted robot is mounted on casters.

In accordance with a second aspect of the present disclosure, there is provided a system for tracking relative movement between a tracking system and a surgical robot, comprising: a processing unit; and a non-transitory computer-readable memory communicatively coupled to the processing unit and comprising computer-readable program instructions executable by the processing unit for: tracking part of a robot arm of the surgical robot with the tracking system during a surgical procedure, detecting a relative movement between the tracking system and the surgical robot from at least one calibration device scanning the floor, the at least one calibration device mounted to a base of the surgical robot and/or to a support of the tracking system, quantifying the relative movement using data from the at least one calibration device, and correcting and outputting the tracking of the part of the robot arm of the surgical robot with the tracking system as a function of the quantifying of the relative movement.

Further in accordance with the second aspect, for example, detecting and quantifying the relative movement includes detecting and quantifying movement of the base of the surgical robot and/or of the support of the tracking system in translation relative to a plane of the floor.

Still further in accordance with the second aspect, for example, detecting and quantifying the relative movement includes detecting and quantifying movement of the base of the surgical robot and/or of the support of the tracking system in rotation relative to a yaw axis of the base and/or of the support.

Still further in accordance with the second aspect, for example, detecting and quantifying the relative movement includes detecting and quantifying movement of the base of the surgical robot and/or of the support of the tracking systemin rotation relative to a roll axis and/or to a pitch axis of the base and/or of the support.

Still further in accordance with the second aspect, for example, the system may alert a user of the detecting of the relative movement.

Still further in accordance with the second aspect, for example, the system may require the user to validate the quantifying.

Still further in accordance with the second aspect, for example, the system may pause the tracking between the detecting and the qualifying, and resume the tracking after the quantifying.

Still further in accordance with the second aspect, for example, detecting the relative movement includes continuously monitoring the position and orientation of the base of the surgical robot and/or of the support of the tracking system.

Still further in accordance with the second aspect, for example, outputting the tracking includes outputting the tracking graphically on a graphic-user interface.

Still further in accordance with the second aspect, for example, the surgical robot, the tracking system and the at least one calibration device are included in the system.

Still further in accordance with the second aspect, for example, the at least one calibration device includes: a light source configured for projecting light on a floor, an image capture component configured for capturing light reflected and/or backscattered from the floor, and a calibration module having a processing unit and a non-transitory computer-readable memory communicatively coupled to the processing unit and comprising computer-readable program instructions executable by the processing unit for quantifying a movement of the floor-mounted base relative to the floor using data from the image capture component associated with the light reflected and/or backscattered from the floor.

### DESCRIPTION OF THE DRAWINGS

FIG. 1 is a schematic view of a robot-assisted surgery (RAS) system with optical navigation in accordance with the present disclosure;

FIG. 2 is a block diagram of a calibration device as used in the robot-assisted surgery system of FIG. 1; and

FIG. 3 is a flowchart showing an exemplary method for tracking movement of a robot during robot-assisted surgery.

### DETAILED DESCRIPTION

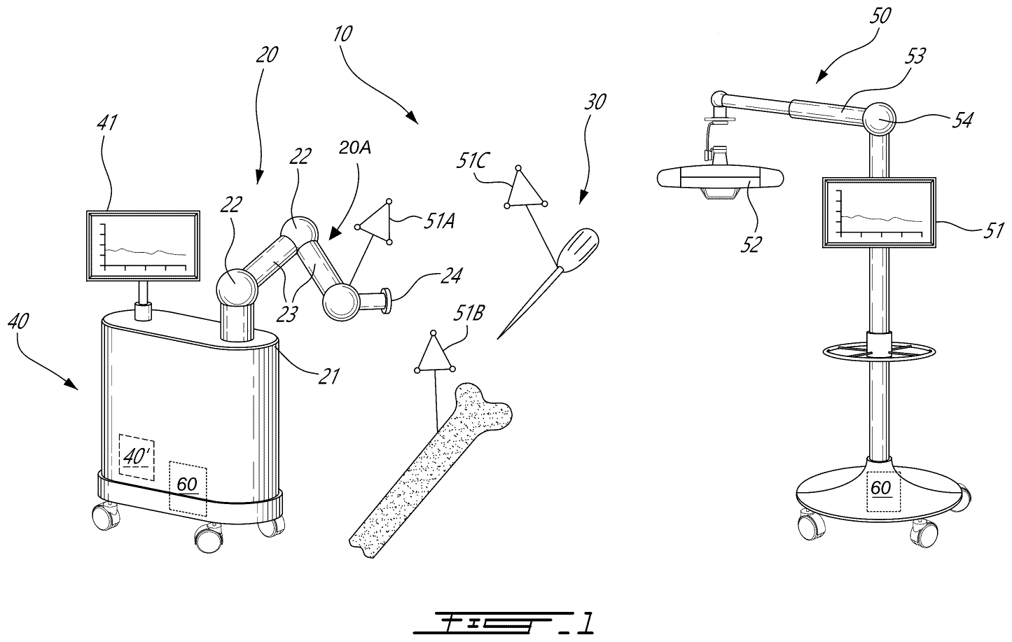

Referring to the drawings and more particularly to FIG. 1, an exemplary robot-assisted surgery (RAS) system is generally shown at 10, and is used to perform orthopedic surgery maneuvers on a patient. Orthopedic surgery is one of numerous types of patient procedures that may be assisted with the RAS system 10, and is merely given as an example as other types of procedures may benefit from computer-assisted navigation. The RAS system 10 may consequently have one or more processing units dedicated to operating a workflow of a surgical procedure. The RAS system 10 may therefore include a non-transitory computer-readable memory communicatively coupled to the one or more processing units and may have computer-readable program instructions executable by the one or more processing units to operate the workflow described herein. In an embodiment, the RAS system 10 drives a surgical robot 20 used autonomously, and/or as an assistive or collaborative tool for an operator (e.g., surgeon). When the RAS system 10 is used as a collaborative tool, the robot arm may be manipulated by a human operator (e.g., a surgeon), with the robot arm operating to limit movement(s), hold a position, etc, with the human operator tasked with “pulling the trigger” to actuate tools supported or guided by the robot arm. This is just an example of a possible collaborative mode. In another embodiment, the system 10 is one used without robotic assistance, and assists operator by way of surgical navigation.

The RAS system 10 is shown relative to a patient's bone such as a tibia or femur, shown schematically, but only as an example. The system 10 could be used for other body parts, including non-exhaustively hip joint, spine, and shoulder bones, or in other applications, including dentistry, craniomaxillofacial, other non-orthopedic surgeries, etc.

The RAS system 10 may include the surgical robot 20, one or more surgical tools 30 such as a digitizer used in hand mode, a RAS controller as part of a station, shown as RAS controller 40, a tracking system 50, and a calibration device(s) 60:
The surgical robot 20 is optionally present, and is one possible working end of the system 10. The surgical robot 20 may be used to perform bone alterations as planned by an operator and/or the RAS controller 40 and as controlled by the RAS controller 40. The surgical robot 20 may support a tool that is operated by personnel, such as a surgeon;
The surgical tool(s) 30 are tools that are manipulated by the operator and/or by the surgical robot 20 in order to perform tasks on the patient. A digitizer is shown in FIG. 1, but other tools may be used depending on the nature of the procedure. Tools 30 may include awls, drills, impactors, saws, to name a few of the one or more tools 30 that may be used, as numerous other tools may be used. The surgical tool(s) may or may not be used depending on whether a surgical robot 20 is in the system 10. It is contemplated to have both the surgical robot 20 and the surgical tool(s) 30 in the RAS system 10;
The RAS controller 40 operates the surgical workflow and controls the surgical robot 20 if present. The RAS controller 40 may also guide an operator through the surgical procedure, by providing intraoperative data of position and orientation, and may therefore have the appropriate interfaces such as mouse, foot pedal, touchscreen, etc;
The tracking system 50 may be used to track the bones and/or soft tissue of the patient, the surgical robot 20 and surgical tools 30 if present. For example, the tracking system 50 may assist in performing the calibration (or registration) of the patient bone with respect to the surgical robot 20, for subsequent navigation in the X, Y, Z coordinate system. The tracking system 50 includes a navigation image-capture device or like imaging device, as detailed below;
The calibration device 60 may be used to track movement of the surgical robot 20 and/or of the tracking system 50. The calibration device 60 may quantify the movement, whether in position and/or orientation, to allow the RAS controller 40 to adjust its actions in real-time or quasi real-time.

The system 10 may be without the surgical robot 20, with the operator performing manual tasks. In such a scenario, the RAS system 10 may only have the tools 30, the RAS controller 40, and the tracking system 50, and could be referred to as a computer-assisted surgery (CAS) system, and not a RAS system. The RAS system 10 may also have non-actuated foot support and thigh support to secure the limb.

Still referring to FIG. 1, a schematic example of the surgical robot 20 is provided. The surgical robot 20 may have a robot arm 20A stand from a base 21, for instance in a fixed relation relative to the operating-room (OR) table supporting the patient. The fixed positioning of the surgical robot 20 relative to the patient is a determinative factor in the precision of the surgical procedure, whereby the base 21 may have its casters (shown) or any other support structure locked into place so as not to move. The robot arm 20A has a plurality of joints 22 and links 23, of any appropriate form, to support a tool head 24 that interfaces with the patient or with a tool. The tool head 24 may be an appropriate type of tool and may be interchangeable, as a function of the nature of the surgical procedure. In particular, the tool head 24 may be a clamp that supports a tool, such as those shown at 30, with the tools 30 being manipulated and/or actuated by a human operator.

The robot arm 20A of the surgical robot 20 is shown being a serial mechanism, arranged for the tool head 24 to be displaceable in a desired number of degrees of freedom (DOF). For example, the surgical robot 20 controls movements of the tool head 24. In an embodiment, the robot arm 20A is a 6-DOF articulated arm, i.e., X, Y, Z in the coordinate system, and pitch, roll and yaw. Fewer or additional DOFs may be present. For simplicity, only a generic illustration of the joints 22 and links 23 is provided, but more joints of different types may be present to move the tool head 24 in the manner described above. The joints 22 are powered for the robot arm 20A to move as controlled by the controller 40 in the six DOFs. Therefore, the powering of the joints 22 is such that the robot arm 20A may execute precise movements, such as moving along a single direction in one translation DOF, or being restricted to moving along a plane, among possibilities. Such surgical robots 20 are known, for instance as described in U.S. patent application Ser. No. 11/610,728 and Ser. No. 12/452,142, incorporated herein by reference.

The RAS controller 40 has a processor unit 40′ (a.k.a., processing unit, such as a processor, CPU, ASIC, etc) to control movement of the surgical robot 20, if applicable. The RAS controller 40 provides computer-assisted surgery guidance to an operator, whether in the form of navigation data, model assessment, or in pre-operatively planning or during the surgical procedure. The RAS controller 40 is shown being integrated into the housing in the base 21 of the surgical robot 20, but may be at other locations, for example if the RAS system 10 does not have the surgical robot 20. The RAS controller 40 may be a self-standing computer (e.g., laptop, tablet, etc) or may be integrated or supported in the base of the tracking system 50. The system 10 may comprise various types of interfaces, for the information to be provided to the operator, for instance via the graphic-user interfaces (GUIs) 41 and/or 51. The GUI 41, 51 may be monitors and/or screens including wireless portable devices (e.g., phones, tablets, augmented reality headsets), audio guidance, LED displays, among many other possibilities. The RAS controller 40 may drive the surgical robot 20 in performing the surgical procedure based on a pre-operative or peri-operative planning or through operator guidance intra-operatively. The RAS controller 40 runs various modules, in the form of algorithms, code, non-transitory executable instructions, etc, in order to operate the system 10 in the manner described herein. For example, the RAS controller 40 integrates non-transitory computer-readable memory communicatively coupled to the processing unit 40′ and comprising computer-readable program instructions executable by the processing unit 40′. Stated differently, the RAS controller 40 integrates a non-transitory computer-readable medium storing instructions that, when executed by a processor, cause the processor to perform operations.

The use of the tracking system 50 may provide tracking data to perform surgical navigation. For example, the tracking system 50 may assist in performing the calibration of the patient bone with respect to the coordinate system (i.e., the locating of the bone in the coordinate system), for subsequent navigation in the X, Y, Z coordinate system. According to an embodiment, the tracking system 50 comprises an image capturing device 52, also known as a navigation camera, that optically sees and recognizes references 51A, 51B, and 51C (e.g., retro-reflective references, optically recognizable references)—that may be part of the tools used in surgery—, so as to track the robot arm 20A of the surgical robot 20 and/or one or more tools 30 and limbs in six DOFs, namely in position and orientation. In an embodiment featuring the surgical robot 20, the reference 51A is on the tool head 24 of the surgical robot 20 such that its tracking allows the RAS controller 40 to calculate the position and/or orientation of the tool head 24 and/or of any tool thereon. Likewise, references 51B and 51C are fixed to the patient bones, such as the tibia for reference 51B and the femur for reference 51C. In an embodiment without the surgical robot 20, references such as reference 51A are on the navigated tools 30 (including a registration tool shown) such that their tracking allows the controller 40 to calculate the position and/or orientation of the tools and register points. Likewise, references 51B may be fixed to the patient bones. The references 51A-C attached to the patient need not be invasively anchored to the bone, as straps or like attachment means may provide sufficient grasping to prevent movement between the references 51A-C, and the bones, in spite of being attached to soft tissue. However, the references 51B and 51C could also be secured directly to the bones. Therefore, the controller 40 continuously updates the position and/or orientation of the surgical robot 20, tools 30 and patient bones in the X, Y, Z coordinate system using the data from the tracking system 50.

Although the set of references 51A, 51B, 51C and image-capture device 52 is one of type featuring retro-reflective spheres (e.g., Navitrack® system) or tokens such as in U.S. Pat. No. 8,386,022, other image based tracking technologies may be used, such as depth cameras, 3D cameras, etc, without the need for trackers or like trackable references on objects, or with other types of trackers, such as QR codes, etc. The use of the expression “image-capture device” herein is deemed to incorporate all such imaging devices using for navigation. The image-capture device 52 is configured to generate an image, such as image data, of a field of view of the image-capture device 52. In some implementations, a controller or processor of the image-capture device 52 is configured to perform one or more operations based on or using the image data, such as one or more image processing functions. For example, the one or more operations may include object detection, object recognition, object tracking. Additionally, or alternatively, the image-capture device 52 may be configured to generate an output, such as the image data or an indicator of an operation or a result of an option. Moreover, even though the expression “image-capture device” may be used in the singular, the image-capture device 52 may include more than a single point of view, for example using triangulation as an option with two points of view.

In an embodiment, the image-capture device 52 is mounted onto a stationary structure, such as a ground stand, which may include the GUI 51, and a processor unit (optionally present, but that could be the processor unit 40 if no surgical robot 20 is present). An articulated arm featuring links 53 and joints 54 may optionally be present, with the joints 54 being lockable or being capable of maintaining a set position and orientation, whereby the articulated arm may be regarded a stationary structure. This may allow an adjustment of the orientation of the image-capture device 52, as a line of sight may be required between the image-capture device 52 and the objects being tracked in surgery. For example, the image-capture device 52 may stand over the patient and look down on the surgical zone. The stand may be on casters or like supporting structure to fix the stand in position on the ground. It is also contemplated to use other types of structures or mechanisms to support the image-capture device 52, such a ceiling-mounted arm, a wall-mounted arm, a table mounted arm, a console-mounted arm, etc.

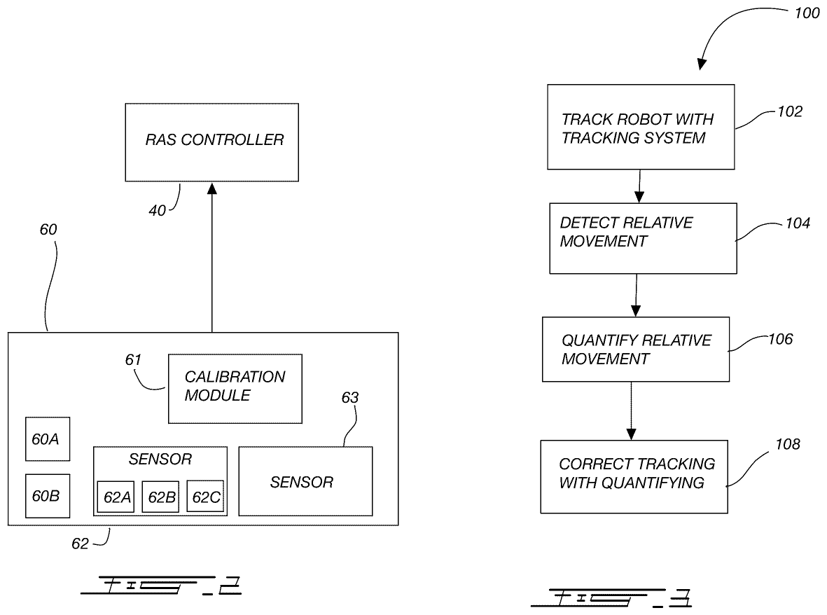

Referring concurrently to FIGS. 1 and 2, the calibration device 60 may include one or more processors 60A and a non-transitory computer readable memory 60B coupled communicatively to the processor 60A, whether the processor 60A be part of the RAS controller 40 (e.g., the processor 60A could be the processor 40′) and shared with the calibration device 60 or dedicated to the calibration device 60. Likewise, the non-transitory computer readable memory may be dedicated to the calibration device 60, or may be shared or part of the RAS controller 40. The calibration device 60 may further include computer readable memory program instructions executable by the processor, in the form of a calibration module 61, for example. The calibration device 60 may be used to monitor movement of the base 21 of the robot 20 and/or of the tracking system 50. The calibrating may be effected in real-time or quasi-real-time. For example, the calibration device 60 may be retrofitted to existing surgical robots and/or to tracking systems.

The calibration module 61 will operate using signals from different sensors.

The calibration device 60 may include a planar displacement sensor(s) 62. In a variant, the planar displacement sensor 62 may be an image acquisition system that is in close proximity to the ground, for example by having the planar displacement sensor 62 located at the bottom wall of the base 21 of the robot 20, and in a similar position on a base of the tracking system 50. For simplicity, reference is made herein to the calibration device 60 as used with the robot 20, but the calibration device 60 may be used in a similar manner with the tracking system 50, if present, or if the tracking system 50 has a calibration device 60.

According to an embodiment, the planar displacement sensor(s) 62 includes a light source 62A, lenses 62B and an image capture component 62C (e.g., charge-coupled device), such as an image pixel array. The light source 62A may be a LED (light emitting diode) light source (e.g., infrared), a laser light source, as examples among others. Lenses 62B may optionally be present to orient the light from the light source 62A onto the floor, and to then focus reflected and/or backscattered light onto the image capture component 62C. The reflection may be specular reflection and/or diffuse reflection depending on the surface type. Moreover, light may be backscattered, for instance if some particles are on the floor. For simplicity, reference is made herein to reflected and/or backscattered light to cover light returning toward the base 21 of the robot 20 or the base of the tracking system 50, whether the light is from specular reflection, diffuse reflection and/or backscatter. In a variant, one of the lenses 62B redirects the light from the light source 62A at a shallow angle onto the floor. As a result, the angle of the light may expose the texture of the floor surface. The light that is then captured by the image capture component 62C, such as via another lens 62B, is representative of the texture of the floor surface. The floor surface is therefore used as a finger print, and the frequency of image capture by the image capture component 62C is such that the position of the planar displacement sensor(s) 62 relative to the floor plane can be updated in real-time. In a variant, the planar displacement sensor 62 is at a distance of at most 6 inches from the ground. This may mean that the light source 62A and/or one or more of the lenses 62B (e.g., the one that is closest to the floor) and/or the image capture component 62C is at a distance of at most 6 inches to the floor. Thus, the planar displacement sensor(s) 62 can update its position relative to the floor. Depending on how fast the planar displacement sensor(s) 62 is moving, each image captured by the image capture component 62C will be offset from the previous one if there is movement, such as on the pixels of the image capture component 62C. The calibration module 61 may process these images from the image capture component 62C using cross correlation to calculate how much each successive image is offset from the previous one, and therefore provide X and Y displacement values, as well as a variation about a yaw axis. The processor 60A may have all components to achieve the calculation, such as a digital signal processor for the cross correlation calculation. The image capture component 62C is selected to have sufficient resolution to satisfy precision requirements associated with computer-assisted surgery. The planar displacement sensor(s) 62 essentially scans the floor.

As the planar displacement sensor(s) 62 is secured to the base 21 of the robot arm 20, the movement of the planar displacement sensor(s) 62 relative to a plane of the floor, i.e., a X-Y plane, can be quantified and is representative of the movement of the base 21 of the robot 20. Thus, if the robot 20 is located at an initial X-Y position relative to the floor, which initial X-Y position is monitored, the calibration device 60, via the planar displacement sensor(s) 62, may quantify any X-Y translational movement and/or a rotation about the yaw axis (normal to a plane of the floor), and output a value of movement, representative of the displacement relative to the initial X-Y position and/or to the initial orientation in the X-Y plane. The RAS controller 40 may thus take this displacement in consideration when using the tracking data from the tracking system 50, as the relative position between the robot 20 and the tracking system 50 has changed through this displacement, and the tracking system 50 performs the tracking based on a current relative position between the robot 20 and the tracking system 50.

Other examples that may be used instead of the planar displacement sensor(s) 62 may include an ultrasound sensor(s) and/or an optical sensor(s) that may determine a X-Y displacement and yaw axis rotation. Ultrasound sensor(s) may also be suited to perform the position sensing considering that the base 21 of the robot 20 is always in close proximity to support surfaces and hence can echo soundwaves emitted by ultrasound sensor(s). The ultrasound sensor(s) is deemed to be an integrated solution, including an emitter and a receiver, as well as the processing circuitry to interpret echo signals. Part of the processing may also be done through the process of the calibration device 60.

Other types of sensors may be used as well to perform such functions. The sensors 62 and/or 63, if present, are communicatively coupled to the calibration module 61 such that the calibration module 61 receives signals from the sensors 62 and/or 63 and interprets them to quantify movement of the base 21 of the robot 20 relative to the floor, and/or of the base of the tracking system 50 relative to the floor, if a calibration device 60 is on the tracking system 50.

In an embodiment, the calibration device 60 has an incline sensor 63 or set of sensor(s) 63, that may also be known as orientation sensor(s). Such sensor(s) 63 detects angular variations or angular rates of change. The incline sensor(s) 63 is tasked with monitoring angular variations of the base 21 of the robot 20 and/or of the base of the tracking system 50 relative to the floor, including at least the rotation about the pitch axis and/or roll axis. In an embodiment, the incline sensor(s) 63 includes one or more inertial sensors as their sourceless nature is well suited for use in the calibration device 60. For example, the incline sensor(s) 63 may include one or more of an gyroscope, an accelerometer and/or an inclinometer, and/or combinations thereof, or other MEMS (micro-electromechanical systems). One or more of the sensors 63 may be used in conjunction with an internal clock or like time monitoring feature, etc. It is contemplated to have numerous sensors 63 of one or more types in order to provide redundancy to the calibration device 60.

In a variant, the planar displacement sensor(s) 62 is at the bottom of the robot 20, continuously scanning the floor during use of the robot 20, to measure any planar displacement along the floor, and rotation in the yaw axis. If the assumption is made that the floor is level, rotations about the roll axis and/or the pitch axis may not occur. However, the sensor(s) 63 may be used to monitor any orientation about the roll axis and/or the pitch axis.

Likewise, in a variant, the planar displacement sensor(s) 62 is at the bottom of the tracking system 50, continuously scanning the floor during use of the tracking system 50, to measure any planar displacement along the floor, and rotation in the yaw axis. Again, if the assumption is made that the floor is level, rotations about the roll axis and/or the pitch axis may not occur. However, the sensor(s) 63 may be used to monitor any orientation about the roll axis and/or the pitch axis.

In a variant, a reference tracker, such as reference trackers 51A-51C, may be provided on the robot base 21, or the position and/or orientation of the robot base 21 may be calculated by tracking the end effector 24 of the robot arm and using encoder signals from the joints in the robot arm to determine the position and/or orientation of robot base 21, as redundant information, and vice versa. Then, in situations where line of sight to the reference 51A of the robot arm is compromised, the position and orientation of the end effector 24 of the robot arm may be calculated by using the known arm position and orientation and the data from the calibration device 60.

If present in the robot 20, the calibration device 60 may be tasked with determining and recording a position and/or orientation of the robot 20 relative to the floor, such as an initial position and orientation (yaw axis). The calibration module 61 therefore receives signals from the sensors 62 and/or 63, for example throughout a surgical procedure, to detect any displacement, and to calculate a real-time position and/or orientation of the robot base 21 after displacement, and possibly during the displacement. Consequently, if a position and/or orientation of the robot 20 changes during the surgical procedure, the change may be quantified by the calibration device 60. The RAS processor 40 may adjust navigation data using the quantified values of change of position and/or orientation.

In an embodiment, the calibration module 61 of a calibration device 60 in a robot base 21 records a first position and orientation of the robot base 21 relative to the floor. The first position and orientation may be the initial position and orientation, i.e., prior to or at the commencement of a surgical workflow, or before or at a calibration or creation of the global referential system. The recordation may be described as setting and storing the first position and orientation as reference value, for example as angular values in three axes, and X, Y positions. The recordation by the calibration module 61 may in an example be triggered by a user, such as via an interaction using the surgical workflow. The user may proceed with the trigger of recordation after having placed the robot base 21 correctly relative to the operative scene. In another embodiment, the recordation is automatically triggered by the surgical workflow after a given step is reached. One condition may be the stability of the robot base 21. As the global referential system may be a function of the initial position and orientation of the robot 20, its recordation by the calibration device 60 may serve to replicate the initial orientation if an orientation change occurs inadvertently (e.g., by accidental contact).

In an embodiment, the RAS controller 40 may receive the quantification of angular variation from the calibration module 61, and may adjust tracking data as a function of the adjusted position and/or orientation of the robot 20. Stated differently, using the quantified position/orientation variation from the initial orientation to the uncalibrated position/orientation, the RAS controller 40 may adjust the position and orientation of the base 21 of robot 20 relative to the tracking data from the tracking system 50. Stated differently, the RAS controller 40 may take into account a new position/orientation of the robot 20 relative to the tracking system 50 in the triangulation calculations.

In another embodiment, the calibration module 61 is provided in the tracking system 50, and records a first position and orientation of the tracking system 50 relative to the floor. The first position and orientation may be the initial position and orientation, i.e., prior to or at the commencement of a surgical workflow, or before or at a calibration or creation of the global referential system. The recordation may be described as setting and storing the first position and orientation as reference value, for example as angular values in three axes, and X, Y positions. The recordation by the calibration module 61 may in an example be triggered by a user, such as via an interaction using the surgical workflow. The user may proceed with the trigger of recordation after having placed the tracking system 50 correctly relative to the operative scene. In another embodiment, the recordation is automatically triggered by the surgical workflow after a given step is reached. One condition may be the stability of the tracking system 50. As the global referential system may be a function of the initial position and orientation of the tracking system 50, its recordation by the calibration device 60 may serve to replicate the initial orientation if an orientation change occurs inadvertently (e.g., by accidental contact).

In an embodiment, the RAS controller 40 may receive the quantification of angular variation from the calibration module 61, and may adjust tracking data as a function of the adjusted position and/or orientation of the tracking system 50. Stated differently, using the quantified position/orientation variation from the initial position and orientation to the uncalibrated position/orientation, the RAS controller 40 may adjust the position and orientation of the objects from the tracking data provided by the tracking system 50, at its new point of view. Stated differently, the RAS controller 40 may take into account a new position/orientation of the tracking system 50 in the triangulation calculations.

In a variant, the surgical robot 20 may be said to include the calibration device 60. The surgical robot 20 may therefore be described as being for computer-assisted surgery, and may include a floor-mounted base. A robotic arm is supported by the floor-mounted base. A calibration device is on the floor-mounted base. The calibration device may include a light source, an image capture component, lenses for directing light from the light source onto the floor and for directing light reflected and/or backscattered from the floor onto the image capture component. A calibration module is coupled to the image capture component, the calibration module for quantifying a movement of the floor-mounted base relative to the floor using data from the image capture component associated with the light reflected and/or backscattered from the floor.

Referring to FIG. 3, an exemplary method for tracking relative movement between a tracking system and a surgical robot during computer-assisted surgery is generally shown at 100. The method 100 may be performed for example using the RAS system 10, with the surgical robot 20, with the tracking system 50, and with one or more of the calibration device 60. The method 100 may be performed with other types of RAS systems. However, for simplicity, reference is made to components of the RAS system 10.

According to 102, part of a robot arm of the surgical robot is tracked with the tracking system during a surgical procedure. This may include a calibrating step by which a coordinate system, also referred to as a referential system or frame of reference, is created, with the location of the surgical robot and/or the tracking system being recorded in the coordinate system, as initial locations. The recordation may be triggered manually or automatically, via the software, via a button or through the interface, as possibilities. From this point on, navigation may commence, using the tracking system 50, and optionally encoders in the robot arm of the surgical robot.

According to 104, a relative movement between the tracking system and the surgical robot may be detected, from one or more calibration devices scanning the floor, the calibration device mounted to a base of the surgical robot and/or to a support of the tracking system. A user of the system may be alerted of the detecting of the relative movement.

According to 106, the relative movement may be quantified using data from the calibration device(s). The relative movement may be movement of the base of the surgical robot and/or of the support of the tracking system in translation relative to a plane of the floor, and/or movement of the base of the surgical robot and/or of the support of the tracking system in rotation relative to a yaw axis of the base and/or of the support, and/or movement of the base of the surgical robot and/or of the support of the tracking systemin rotation relative to a roll axis and/or to a pitch axis of the base and/or of the support. In a variant, the user may be required to validate the quantifying. In variant, the tracking may be paused between the detecting and the qualifying, and the tracking may resume after the quantifying.

According to 108, the tracking of the part of the robot arm of the surgical robot with the tracking system may be corrected as a function of the quantifying of the relative movement. Outputting the tracking may include outputting the tracking graphically on a graphic-user interface.

Throughout the method 100, the position and orientation of the base of the surgical robot and/or of the support of the tracking system may be continuously monitored to detect relative movement. Thus, the system may take into consideration the quantification of the movement. The quantification of 106 may therefore be used as a corrective factor to calculate tracking data from the new point of view of the tracking system 50. Continuous monitoring may include monitoring without pause at a regular frequency to detect any movement. There may be occasional pauses, for instance for a new patient, etc.

The method 100 may be performed by the RAS controller 40 as part of the system 10. The system 10 may be described as being a system for tracking relative movement between a tracking system and a surgical robot, that may include a processing unit; and a non-transitory computer-readable memory communicatively coupled to the processing unit and comprising computer-readable program instructions executable by the processing unit for: tracking part of a robot arm of the surgical robot with the tracking system during a surgical procedure, detecting a relative movement between the tracking system and the surgical robot from at least one calibration device scanning the floor, the at least one calibration device mounted to a base of the surgical robot and/or to a support of the tracking system, quantifying the relative movement using data from the at least one calibration device, and correcting and outputting the tracking of the part of the robot arm of the surgical robot with the tracking system as a function of the quantifying of the relative movement. Detecting and quantifying the relative movement may include: detecting and quantifying movement of the base of the surgical robot and/or of the support of the tracking system in translation relative to a plane of the floor; detecting and quantifying movement of the base of the surgical robot and/or of the support of the tracking system in rotation relative to a yaw axis of the base and/or of the support; and/or detecting and quantifying movement of the base of the surgical robot and/or of the support of the tracking systemin rotation relative to a roll axis and/or to a pitch axis of the base and/or of the support.

In variants, the system may also: alert a user of the detecting of the relative movement; requiring the user to validate the quantifying; pause the tracking between the detecting and the qualifying, and resuming the tracking after the quantifying; continuously monitor the position and orientation of the base of the surgical robot and/or of the support of the tracking system; and/or output the tracking graphically on a graphic-user interface.

1. A surgical robot for computer-assisted surgery, comprising:
a floor-mounted base;
a robotic arm supported by the floor-mounted base; and
a calibration device on the floor-mounted base or robotic arm, the calibration device including
a light source configured for projecting light on a floor,
an image capture component configured for capturing light reflected and/or backscattered from the floor, and
a calibration module having a processing unit and a non-transitory computer-readable memory communicatively coupled to the processing unit and comprising computer-readable program instructions executable by the processing unit for quantifying a movement of the floor-mounted base relative to the floor using data from the image capture component associated with the light reflected and/or backscattered from the floor.

2. The surgical robot according to claim 1, wherein the calibration module is for quantifying a movement of the floor-mounted base in translation relative to a plane of the floor, and in rotation relative to a yaw axis of the base.

3. The surgical robot according to claim 1, wherein the calibration device further includes an inertial sensor, the calibration module coupled to inertial sensor for quantifying a variation of orientation of the floor-mounted base relative to the floor using data from the inertial sensor, the variation of orientation being relative to a roll axis and/or to a pitch axis of the base.

4. The surgical robot according to claim 1, further including at least one lens for directing light from the light source onto the floor and for directing light reflected and/or backscattered from the floor onto the image capture component.

5. The surgical robot according to claim 4, wherein one of the at least one lens is configured to be at most 6 inches from the floor.

6. The surgical robot according to claim 1, wherein the calibration device is mounted to the floor-mounted base.

7. The surgical robot according to claim 6, wherein the light source and/or the image capture component is at most 6 inches from the floor.

8. The surgical robot according to claim 1, wherein the light source is a LED light source.

9. The surgical robot according to claim 1, wherein the image capture component is an image pixel array.

10. A system for tracking relative movement between a tracking system and a surgical robot, comprising:
a processing unit; and
a non-transitory computer-readable memory communicatively coupled to the processing unit and comprising computer-readable program instructions executable by the processing unit for:
tracking part of a robot arm of the surgical robot with the tracking system during a surgical procedure,
detecting a relative movement between the tracking system and the surgical robot from at least one calibration device scanning the floor, the at least one calibration device mounted to a base of the surgical robot and/or to a support of the tracking system,
quantifying the relative movement using data from the at least one calibration device, and
correcting and outputting the tracking of the part of the robot arm of the surgical robot with the tracking system as a function of the quantifying of the relative movement.

11. The system according to claim 10, wherein detecting and quantifying the relative movement includes detecting and quantifying movement of the base of the surgical robot and/or of the support of the tracking system in translation relative to a plane of the floor.

12. The system according to claim 11, wherein detecting and quantifying the relative movement includes detecting and quantifying movement of the base of the surgical robot and/or of the support of the tracking system in rotation relative to a yaw axis of the base and/or of the support.

13. The system according to claim 10, wherein detecting and quantifying the relative movement includes detecting and quantifying movement of the base of the surgical robot and/or of the support of the tracking systemin rotation relative to a roll axis and/or to a pitch axis of the base and/or of the support.

14. The system according to claim 10, including alerting a user of the detecting of the relative movement.

15. The system according to claim 14, including requiring the user to validate the quantifying.

16. The system according to claim 10, including pausing the tracking between the detecting and the qualifying, and resuming the tracking after the quantifying.

17. The system according to claim 10, wherein detecting the relative movement includes continuously monitoring the position and orientation of the base of the surgical robot and/or of the support of the tracking system.

18. The system according to claim 10, wherein outputting the tracking includes outputting the tracking graphically on a graphic-user interface.

19. The system according to claim 10, including the surgical robot, the tracking system and the at least one calibration device.

20. The system according to claim 19, wherein the at least one calibration device includes:
a light source configured for projecting light on a floor,
an image capture component configured for capturing light reflected and/or backscattered from the floor, and
a calibration module having a processing unit and a non-transitory computer-readable memory communicatively coupled to the processing unit and comprising computer-readable program instructions executable by the processing unit for quantifying a movement of the floor-mounted base relative to the floor using data from the image capture component associated with the light reflected and/or backscattered from the floor.
